(19) United States
(12) Patent Application Publication
(10) Pub. No.: US 2024/0261588 A1
(43) Pub. Date: Aug. 8, 2024

(54) PHOTON BEAUTY MIRROR

(71) Applicant: SHENZHEN YUYI ELECTRONIC TECHNOLOGY CO., LTD., Shenzhen City (CN)

(72) Inventors: YING ZHOU, Shenzhen City (CN); BING LI, Shenzhen City (CN)

(21) Appl. No.: 18/429,391

(22) Filed: Jan. 31, 2024

(30) Foreign Application Priority Data
Jan. 31, 2023 (CN) 202310048031.3
Mar. 20, 2023 (CN) 202310271786.X

(51) Int. Cl. A61N 5/06

(52) U.S. Cl. CPC A61N 5/0616; A61N 2005/0633; A61N 2005/0659; A61N 2005/0665; A61N 2005/0667

(57) ABSTRACT

The present invention provides a photon beauty mirror, comprising: a main frame, a first light source assembly, a circuit board and a special optical filter. The special optical filter comprises an optical substrate coated with a filter film of a preset wavelength bands on one side and coated with a one-way perspective film on an opposite side of the optical sheet, light of the preset wavelength bands passes through the filter film for beauty treatment; the one-way perspective film reflects visible light to obtain a mirror effect; or, comprises an optical substrate coated with a filter film, where near infrared light or non-visible light above 700 nm transmit through the filter film for beauty treat, and the filter film reflects visible to obtain a mirror effect.

### CROSS REFERENCE TO RELATED APPLICATIONS

The present invention is 35 U.S.C. § 119 benefit of earlier filing dates; rights of priority of Chinese Applications No. 202310048031.3 filed on Jan. 31, 2023, and Chinese Applications No. 202310271786.X filed on Mar. 20, 2023, the disclosures of which are incorporated by reference herein.

### BACKGROUND OF THE INVENTION

### Field of the Invention

The present invention relates to the field of beauty instruments, and more particularly, to a photon beauty mirror.

### Description of Related Art

Intense Pulsed Light (IPL) is a broad-spectrum visible light that has achieved remarkable results in the treatment of vascular lesions, pigmented lesions, hirsutism, scars, acne, and rejuvenation. IPL is a higher intensity light source usually with a wavelength of 400-1200 nm, and is filtered using a filter to obtain a specific wavelength band suitable for skin treatment or beauty. The photons of IPL can penetrate into the deeper layers of the skin, and through photothermal and photochemical effects, the elastic fibers and collagen fibers of the skin are rearranged and combined again, which can restore the elasticity of the skin, make the skin tighter and more youthful, and has the effect of improving acne, removing redness, darkening, whitening, diluting fine lines, improving dark skin, and brightening the skin.

For hair removal, skin rejuvenation, whitening, spot removal, redness removal, etc., some current beauty instruments with small light windows (with an area of 1-4 cm²) such as hair removal devices, photon skin rejuvenation devices or beauty masks are used, due to the small light windows, the beauty instrument needs to be moved back and forth to move the light window during use, this will cause the skin to be repeatedly irradiated or not irradiated, resulting in uneven skin color; some current beauty instrument with a large IPL light window will easily damage the skin and tissue; and the current LED mask cannot treat different skin symptoms due to insufficient light source energy and a single fixed wave band.

### BRIEF SUMMARY OF THE INVENTION

An object of the present invention is to provide a photon beauty mirror.

The present invention provides a photon beauty mirror, comprising: a main frame; a first light source assembly disposed in the main frame, and comprising a first light source for emitting light; a circuit board disposed in the main frame and electrically connected with the first light source assembly; and a first optical sheet assembly installed to the main frame comprising one or more optical filters for filtering light from the light source to obtain a preset wavelength bands. Wherein at least one optical filter is a special optical filter. The special optical filter comprises: an optical substrate coated with a filter film of a preset wavelength bands on one side and coated with a one-way perspective film on an opposite side of the optical sheet, light from the light source penetrate the one-way perspective film and the optical substrate, and light of the preset wavelength bands passes through the filter film for beauty treatment; the one-way perspective film transmits incident light on one side and reflects visible light on the other side, and the side reflecting incident visible light gives a mirror effect; or, an optical substrate coated with a filter film, where near infrared light or non-visible light above 700 nm transmit through the filter film for beauty treat, and the filter film reflects visible to obtain a mirror effect.

In some embodiments, more first optical sheet assemblies are provided; the first optical sheet assemblies or optical filters are detachably and replaceably installed to the main frame each for filtering light from the first light source; and the optical sheet assemblies each comprise a regular optical filter, or the special filter film of different wavelength bands, or a lens of a reflecting mirror.

In some embodiments, the regular optical filter or the special optical filter or the lens is equipped in a holder.

In some embodiments, the main frame defines one or more slots for receiving the first optical sheet assembly, and the optical sheet assembly or the optical filter is replaceably installed to the main frame by inserting into the slot.

In some embodiments, the optical sheet assembly is replaceably attached to the main frame by magnetic components installed in the main frame and the holder.

In some embodiments, the photon beauty mirror further comprises one or two auxiliary frames each connected to either side of the main frame; wherein each auxiliary frame is equipped with a second light source assembly and a second optical sheet assembly; the second light source assembly comprises a second light source, and the second optical sheet assembly comprises the special optical filter or a regular optical filter; light from the second light source irradiates through the special optical filter or the regular optical filter for beauty treatment.

In some embodiments, the second optical sheet assembly is installed to the auxiliary frame by inserting into a slot defined in the auxiliary frame, or by magnetic components disposed in the auxiliary frame and the second optical sheet assembly, or fixedly installed to the auxiliary frame; the auxiliary frame defines heat dissipation holes; and the main frame defines heat dissipation holes.

In some embodiments, the auxiliary frame is rotatably connected to either side of the main frame through a rotating shaft; the auxiliary frame rotates around either side of the main frame to open or close a front side of the main frame; and a rotatable angle of the auxiliary frame is adapted for different widths of user's face to be treated.

In some embodiments, the first light source assembly comprises a rotatable holder fixing an end of the one or more optical filters; the optical filter is in a shape of a cylinder or are fixed by the rotatable holder to form a cylinder; the light source is longitudinally arranged in the cylinder; the rotatable holder takes the cylinder to rotate around the light source so as to change a position of the one or more optical filters.

In some embodiments, the first light source assembly comprises at least one reflector and at least one heat dissipation member; the at least one reflector is disposed behind the light source; the at least one heat dissipation member is installed behind the reflector; the at least one reflector forms a slot that allows the optical filter to rotate therethrough, the at least one heat dissipation member forms an opening that allows the optical filter to rotate therethrough.

In some embodiments, the optical filter is formed by coating filter films on a cylindrical optical substrate, the filter films have different wavelength bands in zones, each zone is formed on the cylinder peripheral wall along a longitudinal direction, different zones of different wavelength bands rotate by rotating the rotatable holder, the light source is longitudinally arranged within the optical filter; the rotatable holder takes the optical filter to rotate around the light source so as to switch different zone of the optical filter in a light transmission direct of the light source; the optical filter rotates around the light source to change positions of the zones; and at least one zone forms the special optical filter.

In some embodiments, the one or more optical filters rotate around the light source to switch from a first position to a second position; and the first position is in a light transmission direct; light from the light source passes through the optical filter switched in the first position to obtain a preset wavelength bands for beauty treatment.

In some embodiments, the rotatable holder is an annular disk, the one or more optical filters are installed by the annular disk along circumferences of the annular disks and extends longitudinally.

In some embodiments, the photon beauty mirror comprises a rotation adjustment device, wherein the rotation adjustment device comprises an adjustment knob, a linkage rod, and a gear; the linkage rod and the gear are connected to the adjustment knob and can rotate together; the gear and one annular disk have external teeth that mesh with each other; the adjusting knob rotates to take the rotatable disk rotating through the linkage rod and the gear.

In some embodiments, the adjustment knob is disposed at a position protruding outside from the main frame to facilitate user operation.

In some embodiments, the photon beauty mirror comprises a handle connected to the main frame, wherein a battery and/or a capacitor electrical connected to the circuit board are installed in the handle.

In some embodiments, the first light source assembly comprises a reflector and a convex lens; the reflector is disposed behind the light source, and the convex lens is disposed between the light source and the optical filter, whereby light from the light source is transmitted through the convex lens to irradiate skin evenly.

In some embodiments, the second light source assembly comprises a reflector and a convex lens; the reflector is disposed behind the second light source, and the convex lens is disposed between the second light source and the optical filter, whereby light from the second light source is transmitted through the convex lens to irradiate skin evenly.

In some embodiments, the photon beauty mirror further comprises goggles to protect the user's eyes; wherein the goggles and the main frame are respectively provided with sensors for detecting a distance between the goggles and the light source so that the circuit board controls turning on/off the light source; the goggles has a positioning block for limiting a distance between the goggles and the main frame; the photon beauty mirror comprises a base and a head support frame; the base is provided with an accommodation cavity for storing the goggles and an accommodation cavity for storing the head support frame; the head support frame is used to support user's head to facilitate positioning the face; a connecting frame is provided between the base and the main frame; the connecting frame defines a ventilation channel in air communication with the main frame; and a fan is provided in the connecting frame and in air communication with the ventilation channel.

In some embodiments, the main frame defines heat dissipation holes and is provided with heat dissipation member therein; the main frame defines air channels in air communication with the dissipation holes, where one air channel passes a surface of the optical filter, one air channel passes through the light source; and one air channel passes through a reflector and/or a heat dissipation member.

### BRIEF DESCRIPTION OF THE DRAWINGS

FIG. 1 is a perspective view of a photon beauty mirror in accordance with a first embodiment of the present invention;

FIG. 2 is a perspective view of the photon beauty mirror with filters being pulled out in accordance with the first embodiment of the present invention;

FIG. 3 is a longitudinal cross-sectional view of FIG. 1;

FIG. 4 is a perspective view of the photon beauty mirror with a goggles;

FIG. 5 is an exploded view of FIG. 4;

FIG. 6 is a perspective view of the photon beauty mirror with a first filter mounted magnetically;

FIG. 7 is a perspective view of the photon beauty mirror in accordance with a second embodiment of the present invention;

FIG. 8 is a longitudinal cross-sectional view of FIG. 7;

FIG. 9 is a transverse cross-sectional view of FIG. 7;

FIG. 10 is an exploded view of FIG. 7;

FIG. 11 is a perspective view of the photon beauty mirror in accordance with a third embodiment of the present invention;

FIG. 12 is a longitudinal cross-sectional view of FIG. 11;

FIG. 13 is a transverse cross-sectional view of FIG. 11;

FIG. 14 is a front longitudinal cross-sectional view of FIG. 11;

FIG. 15 is an exploded view of FIG. 11;

FIG. 16 is a longitudinal cross-sectional view of the photon beauty mirror in accordance with a fourth embodiment of the present invention;

FIG. 17 is a transverse cross-sectional view of the photon beauty mirror in accordance with a fourth embodiment of the present invention;

FIG. 18 is a front longitudinal cross-sectional view of the photon beauty mirror in accordance with a fourth embodiment of the present invention;

FIG. 19 is an exploded view of the photon beauty mirror in accordance with a fourth embodiment of the present invention;

FIG. 20 is a front perspective view of the photon beauty mirror in accordance with a fifth embodiment of the present invention;

FIG. 21 is a rear perspective view of FIG. 20;

FIG. 22 illustrates light source of the photon beauty mirror in accordance with the fifth embodiment of the present invention;

FIG. 23 is a longitudinal cross-sectional view of FIG. 20;

FIG. 24 is a transverse cross-sectional view of FIG. 20;

FIG. 25 is an exploded view of FIG. 20;

FIG. 26 is a schematic diagram of an optical filter in accordance with an embodiment of the present invention; and

FIGS. 27 to 30 illustrate various photon beauty mirrors in application of the optical filter of FIG. 26.

### DETAILED DESCRIPTION OF THE INVENTION

Exemplary embodiments of the present invention will be described in more detail below with reference to the accompanying drawings. Although exemplary embodiments of the present invention are shown in the drawings, it should be understood that the present invention may be implemented in various forms and should not be limited to the embodiments below. These embodiments are intended to enable those skilled in the art to understand the present invention more thoroughly.

It will be understood that the followings words used herein is for the purpose of describing particular example embodiments only and is not intended to be limiting. As used herein, the singular forms “a,” “an,” and “the” are intended to include the plural forms as well, unless the context clearly dictates otherwise. The terms “comprise”, “include”, “contain” and “have” are inclusive and thus indicate the presence of stated feature, element and/or part but do not exclude the presence or addition of one or various other features, elements, component, and/or combinations thereof.

Although the terms first, second, third, etc. may be used in the description or claims to describe various elements, components, regions, layers and/or sections, these elements, components, regions, layers and/or sections shall not be referred to as restricted by these terms. These terms are only used to distinguish one element, component, region, layer or section from another region, layer or section. Terms such as “first,” “second,” and other numerical terms when used in the description or claims do not imply a sequence or order unless clearly indicated by the context. Thus, a first element, component, region, layer or section discussed below could be termed a second element, component, region, layer or section without departing from the teachings of the example embodiments.

For convenience of description, spatially relative terms may be used herein to describe the relationship of one element or feature to another element or feature as shown in the figures. These relative terms, such as “inner”, “outer”, “inside”, “outside”, “below”, “under”, “above”, “on”, “top”, “rear”, etc. Such spatially relative terms are intended to cover different orientations of the device in use or operation in addition to the orientation depicted in the figures. For example, if the device in the figures is turned over, elements described as “below” or “under” other elements or features would then be oriented “above” or “on” the other elements or features. The device may be otherwise oriented (rotated 90 degrees or at other orientations) and the spatially relative terms used are interpreted accordingly.

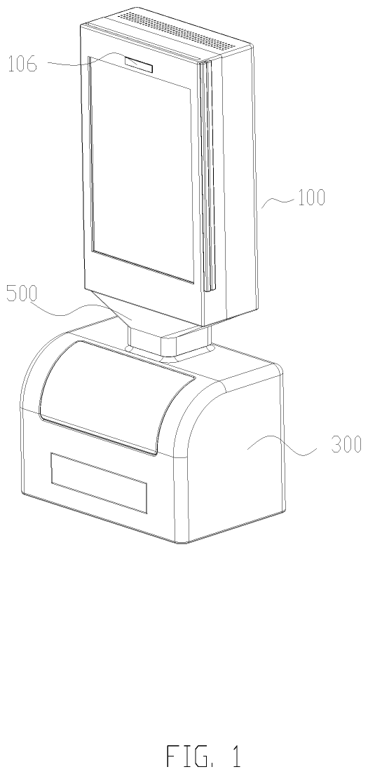

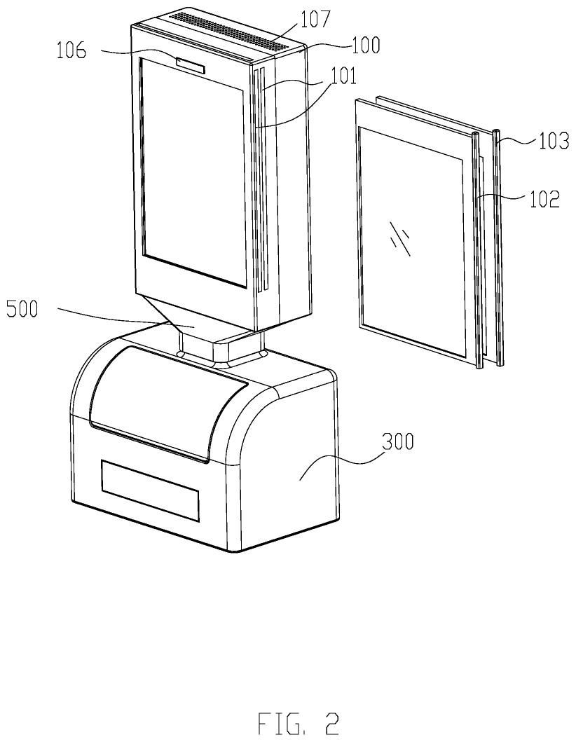

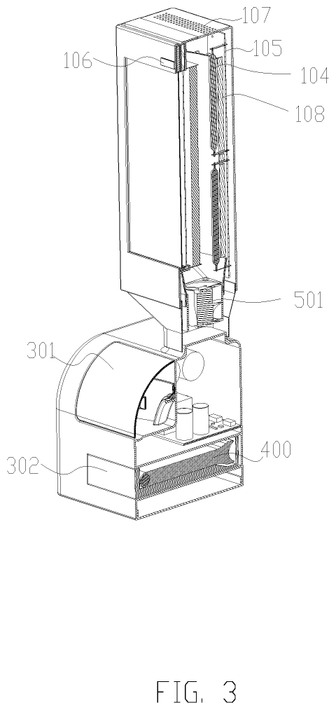

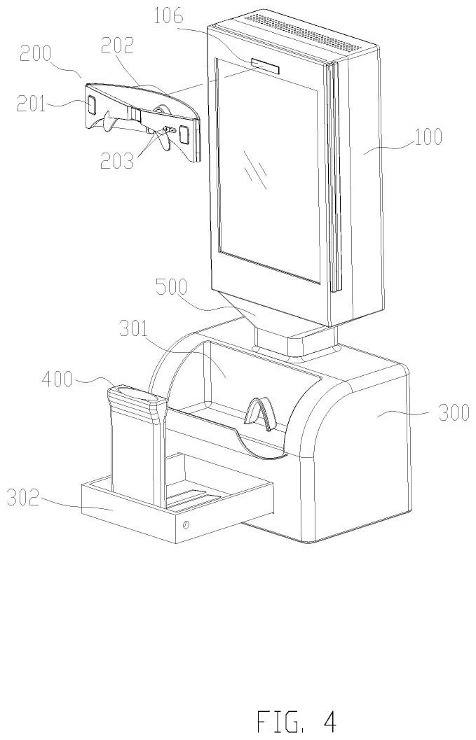

Referring to FIGS. 1 to 4, a photon beauty mirror in accordance with a first embodiment of the invention, comprises a main frame 100, and a first light source assembly, a first optical sheet assembly and a circuit board 105 disposed in the main frame 100. The first light source assembly is connected to the circuit board 105, and light emitted from the first light source assembly irradiates outward through the first optical sheet assembly. Specifically, the first light source assembly includes a light source, and the first optical sheet assembly includes an optical filter. Two or more first optical sheet assemblies are provided to be detachably and replaceable each disposed on or in the main frame 100 which allows optical filters of different wavelength bands to be switched as needed, preferably, large-area filters of the first optical sheet assemblies can be used and replaced, so that it can widely and effectively solve different skin problems corresponding to different skin symptoms. The photon beauty mirror of the present invention uses a large light window (or a large-area filter of the first optical sheet assembly) to cover almost all areas of the face at the same time, including blind spots such as the nose tip and lips. Full-face irradiation will ensure a consistency of facial skin treatment to the greatest extent, greatly improve the overall whitening and rejuvenation effect of facial skin, and greatly shorten use time of the photon beauty mirror.

In some embodiments, the main frame 100 defines two slots 101. The first optical sheet assemblies include a first optical sheet holder 102 and a second optical sheet holder 103 that can be inserted into and accommodated in the two slots 101 respectively. The first optical sheet holder 101 and the second optical sheet holder 102 are respectively equipped with an optical filter. A least one filter of the first optical sheet assemblies 101 has one side coated with a filter film of preset wavelength bands and the opposite side coated with a one-way perspective film; that is, at least one optical filter has the functions of photonic beauty and a cosmetic mirror at the same time. The filter film of the optical filter can be obtained by attaching a metal film to the surface of an optical sheet through a physical or chemical coating process, which can cut off unnecessary light wavelength bands to obtain the purpose of filtering light, so that the light of desired wavelength bands can pass through the filter film and then penetrate the one-way perspective film for beauty treatment. The one-way perspective film transmits incident light on one side and reflects incident light on the other side, and the side reflecting incident light gives a mirror effect. Therefore, when photon beauty function is not used, turn off the light source, and the photon beauty mirror can be used as a regular cosmetic mirror, which effectively improves product utilization.

In another embodiment, one first optical sheet holder 102 or second optical sheet holder 103 is equipped with cosmetic lens, the other is equipped with an optical filter; that is, the filters for photon beauty and the cosmetic lens for a mirror are separately set in different optical sheet holder. The cosmetic lens can give an optical mirror with a high reflectivity. The photon beauty of the present invention can be provided with one or more optical sheet holders. Each optical sheet holder is equipped with an optical filter or a cosmetic lens. The optical filter can be a regular optical filter that transmits light of desired wavelength bands, or can be an optical sheet with a filter film on one side and a one-way perspective film on the other side, where the side with the one-way perspective film can be used as a cosmetic mirror. The optical sheet is made of transparent materials.

The first light source assembly comprises a light source. The light source uses an IPL light source 104, and can further use an LED light source and/or a halogen light source together; for example, can use an IPL lamp together with an LED lamp or LEDs board, or an IPL lamp together with a halogen lamp. The first light source assembly also includes a reflector 1041 and a convex lens 1042. The reflector 1041 is disposed behind the IPL lamp 104 so that light form the light source is transmitted opposite to the reflector 1041, and the convex lens 1042 is disposed between the IPL lamp 104 and an optical filter 109. Light from the light source is transmitted through the convex lens 1042 to irradiate the face evenly.

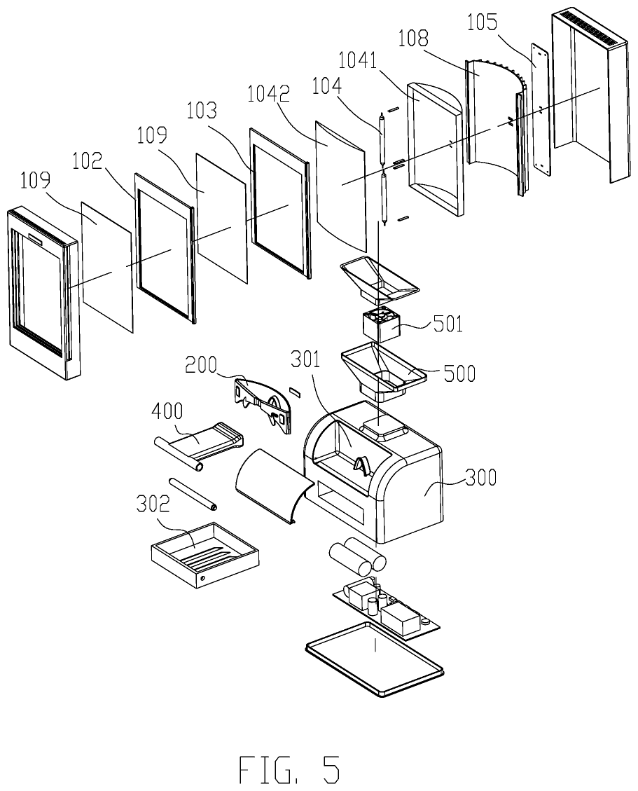

Please refer to FIG. 5 together, goggles 200 can be provided to protect the user's eyes from damaging the eyes when the user use the photon beauty mirror. The goggles 200 and the main frame 100 of the photon beauty mirror are respectively provided with sensors for detecting a distance between the goggles and the light source so that the circuit board 105 controls turning on/off the light source according to the detected signals of the sensor installed on the photon beauty mirror which is electrically connected to the circuit board 105. The light source will be turned on when the goggles (worn on the user's eyes) are detected at a preset distance by the sensor. When the face of the user (wearing the goggles) leaves a preset light-irradiating area, no signal is detected by the sensor of the photon beauty mirror, and the light source is turned off accordingly. If the sensor detects a distance between the face of the user (wearing the goggles) and the light window of the photon beauty mirror too small (exceeding a safe distance), the control circuit 105 controls turning off the light source. In some embodiment, as shown in FIG. 5, the goggles 200 are provided with a sensor 201, and the main frame 100 is provided with a goggle sensing area or sensor 106. A front end and/or both sides of the goggles 200 can be installed with one or more sensors 201.

The goggles 200 are also provided with a positioning block 202 for limiting a distance between the goggles 200 and the main frame 100. When the user's face moves toward the light window of the photon beauty mirror, the positioning block 202 contacts the main frame 100 and so that the user's face (the goggles 200) cannot get close to the mirror to avoid burning. In some embodiments, the goggles 200 are also provided with an adjustment key 203 to adjust the belt of the goggles 200.

The photon beauty mirror includes a base 300 and a head support frame 400. The base 300 is provided with an accommodation cavity 301 for storing the goggles 200 and an accommodation cavity 302 for storing the head support frame 400. A rotation axis can be provided to connect the head support frame 400 so that it can be folded and stored in the accommodation cavity 302 by rotating. The head support frame 400 can be used to support the user's head; the user can position his chin on the head positioning support frame 400 to facilitate positioning the face so that a distance between the face and light window of the photon beauty mirror can keep a fixed distance to avoid facial burns and obtain a precise positioning so that the face is fully exposed to light irradiation.

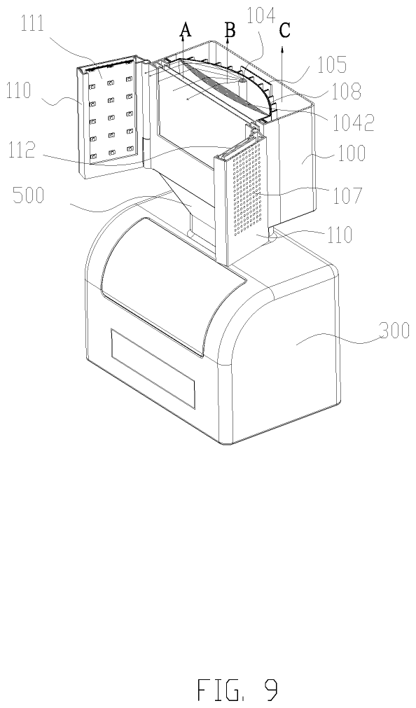

A connecting frame 500 is provided between the base 300 and the main frame 100. The connecting frame 500 defines a ventilation channel in air communication with the main frame 100. A fan 501 is also provided in the connecting frame 500 and in air communication with the ventilation channel. The main frame 100 is provided with heat dissipation holes 107 and heat dissipation member 108. The light source can dissipate heat by a fan and/or by a metal part and/or heat pipes. Please refer to FIG. 9, the main frame 100 defines air channels A/B/C. The air channel A passes a surface of the filter; the air channel B passes through the lamp; and the air channel C passes through the reflector 1041 and heat dissipation member 108.

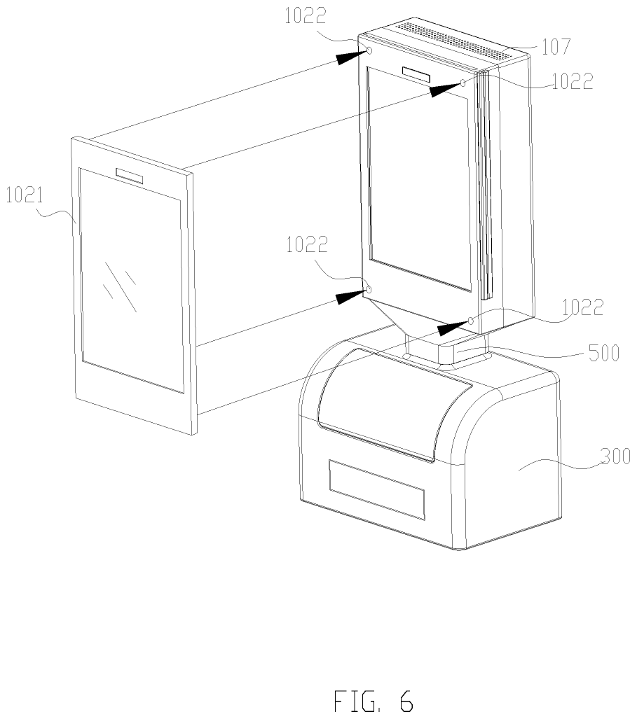

Please refer to FIG. 6, with modifications to the photon beauty mirror in accordance with the first embodiments of FIGS. 1 to 5, one optical sheet assembly 1021 is detachably attached to the main frame 100 by the force exerted by magnetic components. There are one or more optical sheet assemblies 1021 each comprising an optical sheet holder as described above are provided, the optical sheet holder is equipped with an optical filter and/or cosmetic lens as described above. Therefore, the optical sheet assemblies 1021 or the optical sheet holders with optical filters of different wavelength bands and/or cosmetic lens can be selectively attached to the main frame 100. Each optical sheet holder and the main frame 100 of the photon beauty mirror are provided with magnetic components 1022 that attract each other so that the optical sheet holder can be attached and replaced. The one or more optical sheet assemblies 1021 or optical sheet holders can be stored in a back shell of the main frame 100.

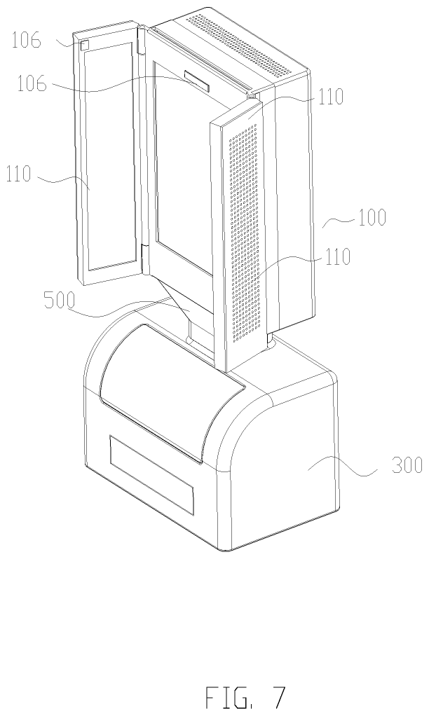

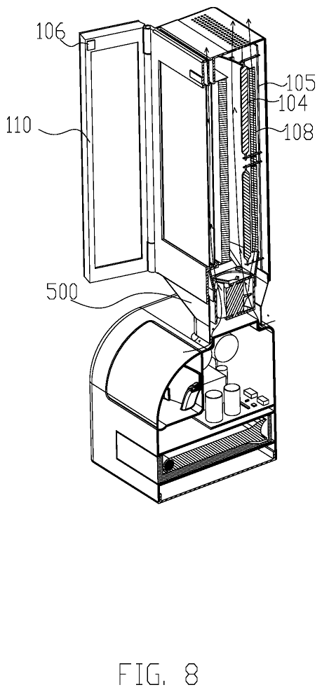

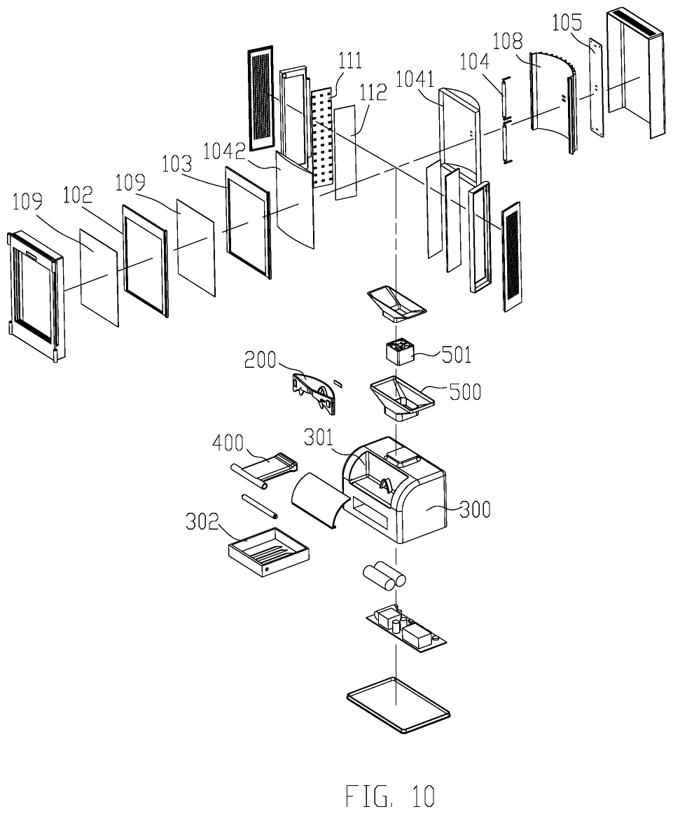

Referring to FIGS. 7 to 10, the photon beauty mirror in accordance with a second embodiment of the present invention is different from the first embodiment in that it further comprises two auxiliary frames 110 each connected to either sides of the main frame 100. Each auxiliary frame 110 is equipped with a second light source assembly and a second optical sheet assembly. The light emitted from the second light source assembly is irradiated outward through the second optical sheet assembly. The light source of the second light source assembly includes one of an IPL light source, an LED light source, or a halogen light source. The second light source assembly in the second embodiment comprises an LED lamp or an LEDs board 111, and an optical filter 112. the second optical sheet assembly may be installed to the auxiliary frame by inserting into a slot in the auxiliary frame 110, or by magnetic components, or fixedly installed to the auxiliary frame 110. The auxiliary frame 110 is also provided with heat dissipation holes 107, and a goggle sensing area or sensor 106.

Light emitted from the first light source assembly through the first optical sheet assembly is transmitted outward from a front side of the photon beauty mirror and can correspondingly irradiate a front face area. Light emitted from the second light source assemblies through the second optical sheet assemblies is transmitted outward from a left side and/or a right side of the photon beauty mirror and can correspondingly irradiate a left face area and/or a right face area.

In the second embodiment, the auxiliary frames 110 are rotatably connected to the main frame 100, and are connected to either sides of the main frame 100 through a rotating shaft respectively, so that the auxiliary frames 110 can rotate around either sides of the main frame 100 to open and close the light window (or the front) of the main frame 100.

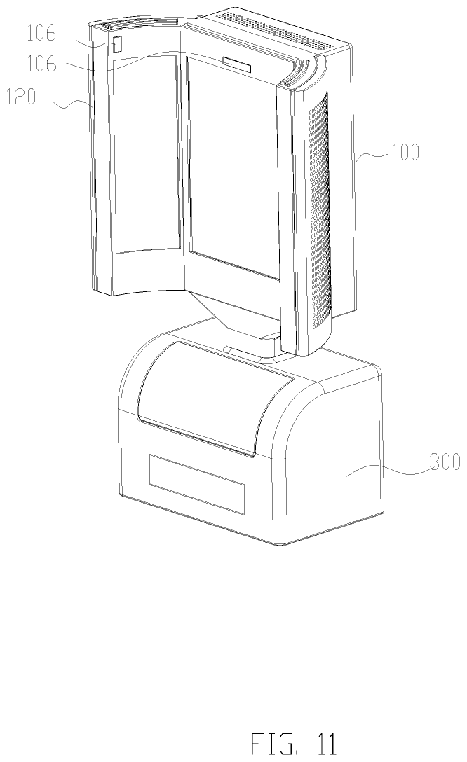

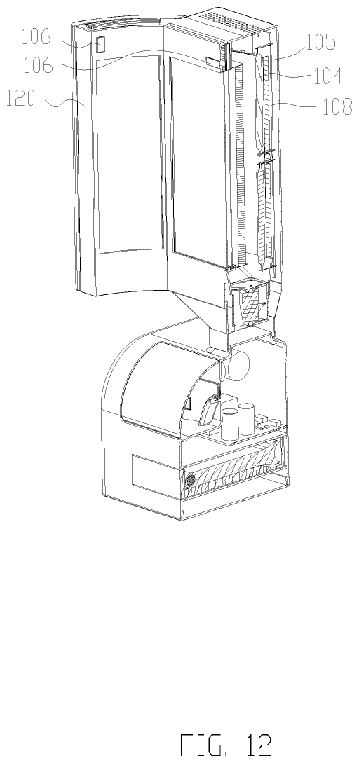

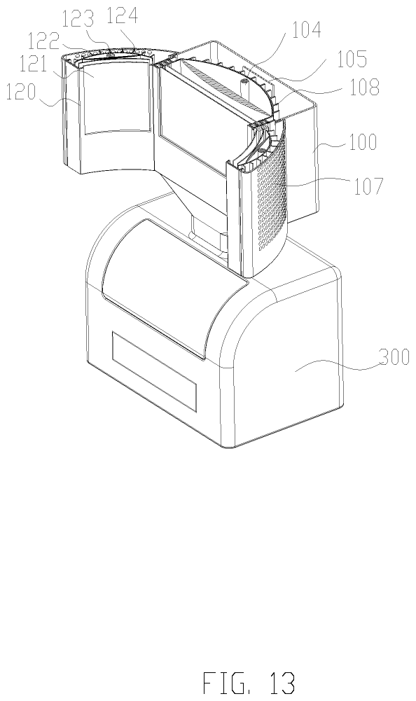

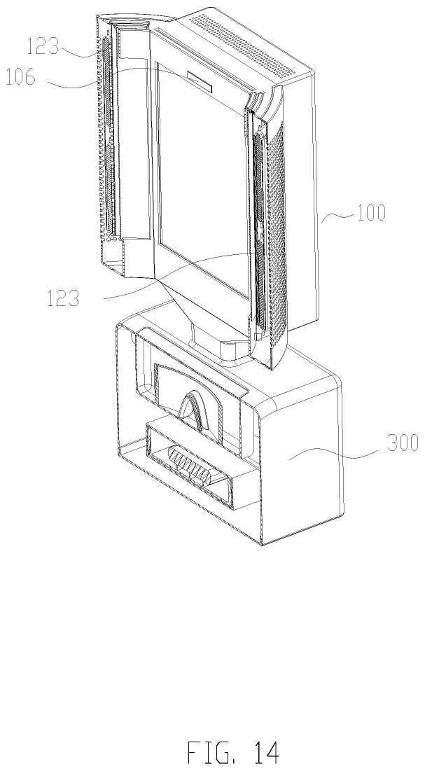

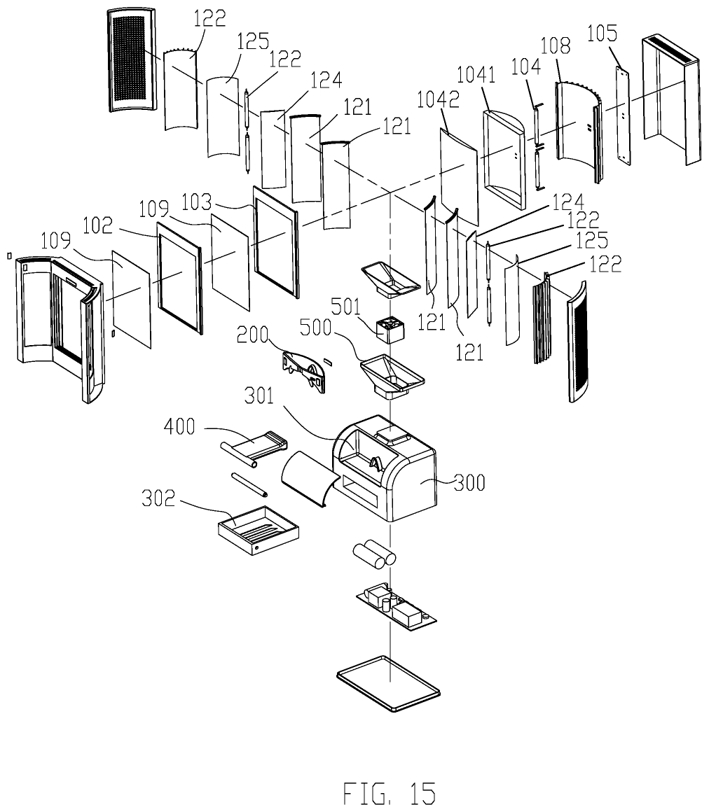

Referring to FIGS. 11 to 15, the photon beauty mirror in accordance with a third embodiment of the present invention is different from the second embodiment in that the second light source assembly of the auxiliary frame 120 includes an IPL light source 123, a convex lens 124 is provided in front of the IPL light source 123, and a reflector 125 and a heat dissipation member 122 are provided behind the IPL light source 123. The second optical sheet assembly includes a third optical sheet holder 121 with an optical filter. The structure of the second light source assembly and the second optical sheet assembly in the auxiliary frame 120 is similar to that of the first light source assembly and the first optical sheet assembly in the main frame 100. The third optical sheet holder 121 can be installed to the auxiliary frame 120 by inserting into a slot in the auxiliary frame 120, or by magnetic components, or fixedly installed to the auxiliary frame 120. The auxiliary frame 120 defines heat dissipation holes 107. The auxiliary frame 120 may also be provided with a goggle sensing area or sensor 106. There are one or more third optical sheet holders 121 provided. The first optical sheet holder 102, the second optical sheet holder 103, and the third optical sheet holder 121 are all examples of the embodiment. In fact, both the main frame 100 and the auxiliary frame 120 can be provided with one or more optical sheet holders. Each optical sheet holder is equipped with an optical sheet. The optical sheet may be an optical filter and/or lens of a cosmetic mirror, or a combination of the two.

The auxiliary frame can be fixedly installed to the main frame 100, or can be installed through a rotatable shaft. A rotatable angle of the auxiliary frame is adapted for different face widths, and/or for storage. The auxiliary frame can also be installed to the main frame 100 in detachable ways such as by buckles, etc.

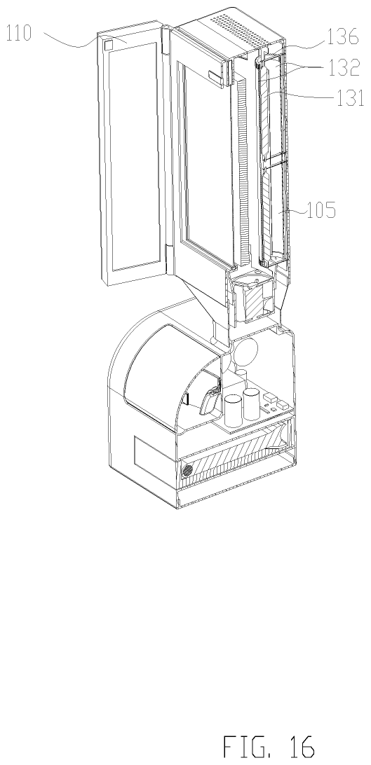

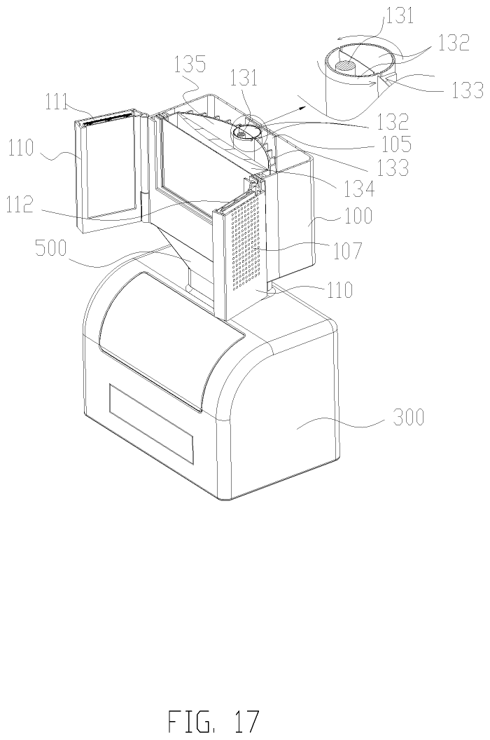

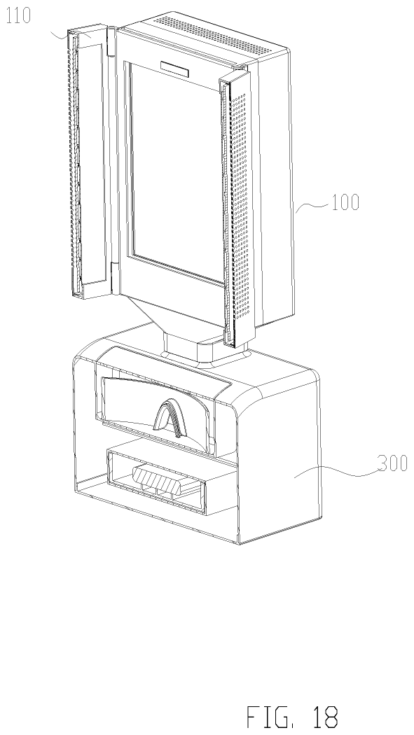

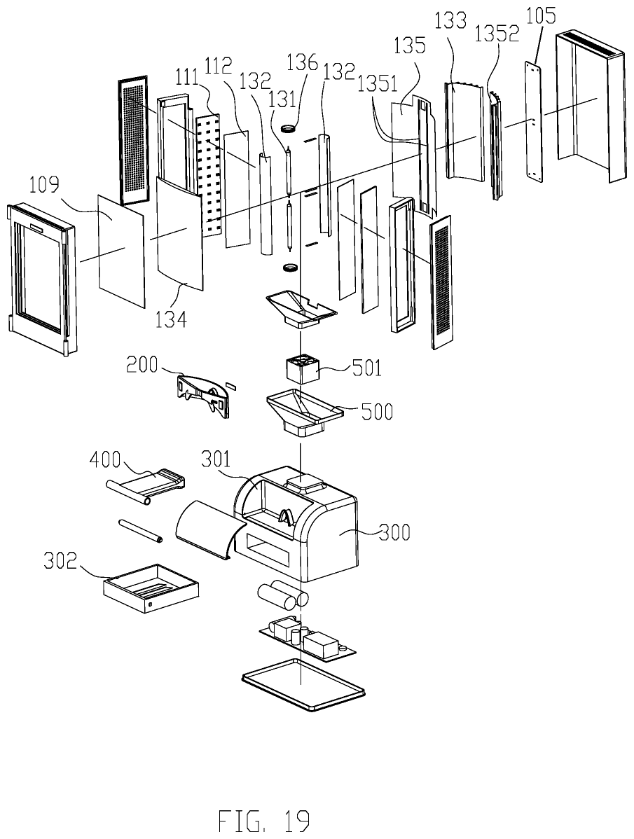

Referring to FIGS. 16 to 19, the photon beauty mirror in accordance with a fourth embodiment of the present invention is different from the second embodiment in that the optical filters in the main frame 100 are arranged in different ways. A first optical sheet assembly is installed in the main frame 100. The first optical sheet assembly includes one or two rotatable holders 136 and two optical filters 132. The two optical filters 132 are fixed by the rotatable holders 136. The rotatable holders 136 rotate around the light source 131 manually or electrically, thus the two optical filters can be rotated around the light source to change positions thereof, thereby the optical filter for the light to be transmitted can be changed. The light source can be a vertically (or axially or longitudinally) arranged IPL lamp. Herein, the two optical filters are only a specific embodiment; one or more optical filters, or a cylindrical optical sheet coated with filter films of different wavelength bands in zones, may be provided. Herein each zone is formed on the cylinder peripheral wall preferably along a longitudinal direction, different zones of different wavelength bands can be switched in the light transmission direction by rotating the rotatable holder 136. The reflector 135 defines a slot 1351 that allows the optical filter 132 to rotate therethrough, and the heat dissipation member 133 also defines an opening 1352 that allows the optical filter 132 to rotate therethrough. Light from the light source 131 is transmitted through the optical filter 132 in the light transmission direction, then is transmitted through the convex lens 134, and is emitted outward through another optical sheet 109. The opening 1352 can be defined by one heat dissipation member 133, or is a space between two heat dissipation members; similarly, slot 1351 can be defined by one reflector 135, or is a space between two reflectors; the slot 1351 and the opening 1352 are adapted for the optical filter 132 and the rotatable holder 136. In this embodiments, each optical filter 132 is semi-cylindrical; two optical filters 132 are fixed by two rotatable holders 136 at opposite ends thereof to form a cylinder with the light source 131 installed therein; the cylinder is rotatable through the slot 1351 of the reflector 135 so that one optical filter 132 is located and the reflector 135 surround the light source 131, and the other optical filter 132 is located between the reflector 135 and heat dissipation member 133. In some embodiments, the optical sheet 109 can be a regular optical filter that transmits light of desired wavelength bands, a cosmetic mirror, or an optical sheet with a filter film on one side and a one-way perspective film on the other side, where the side with the one-way perspective film can be used as a cosmetic mirror. One or more optical sheet holders may also be provided each replaceably installed to the front side of the main frame 100, and each optical sheet holder is equipped with an optical sheet. The optical sheet may be a regular optical filter, lens of a cosmetic mirror, or an optical sheet with a filter film coated on one side and a one-way perspective film coated on the other side.

The photon beauty mirror of the present invention can be powered by an external power through wires or ports, and/or powered by a battery.

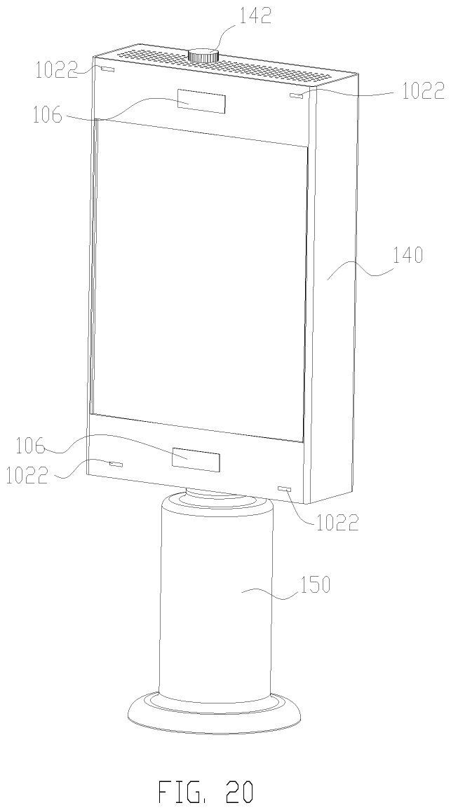

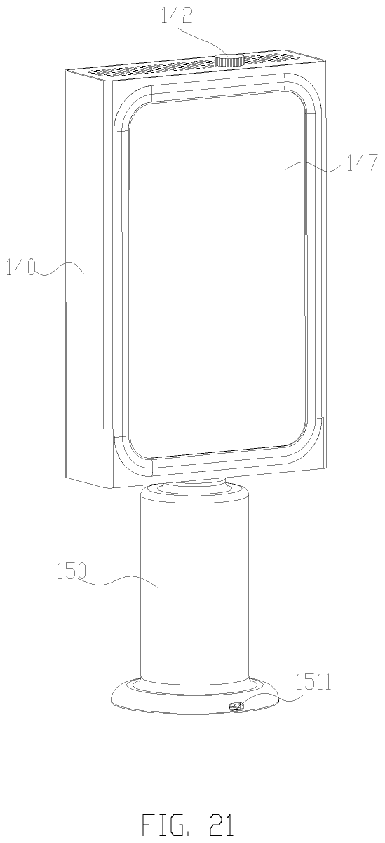

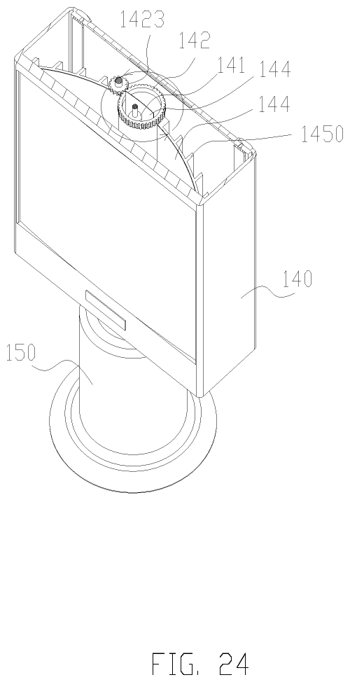

Referring to FIGS. 20 to 25, the photon beauty mirror in accordance with a fifth embodiment of the present invention, comprises a main frame 140, and a first light source assembly, a first optical sheet assembly, and a circuit board 155 arranged in the main frame 140. The first light source assembly is electrically connected with the circuit board 155. Similar to the above-described fourth embodiment, the photon beauty mirror in accordance with the fifth embodiment also uses rotatable filters. As shown in FIGS. 24 and 25, the first light source assembly includes a vertically arranged IPL lamp 141. The optical sheet assembly includes rotatable holders and two optical filters 143 of different wavelength bands. The two optical filters 143 are fixed to the holders. The holders can rotate around the IPL lamp 141 manually or electrically so as to take the two optical filters 143 to rotate around the IPL lamp 141, thereby the two optical filters 143 switch between a first position and a second position respectively around the IPL lamp 141. The first position is located in the transmission direction, and the second position is located opposite to the light transmission direction. Light from the first light source assembly is emitted through the optical filter at the first position, and the optical filter at the first position can be replaced between the two optical filters. Specifically, the two optical filters are only a specific embodiment, and one or more optical filters can be provided. In some other embodiments, if only one optical filter is used, it is switched between the first position and the second position, two states of using or not using the filter are available. In other embodiments, a cylindrical optical sheet coated with filter films of different wavelength bands in zones, may be provided; where each zone is formed on the cylinder peripheral wall preferably along the longitudinal direction, and different zones of different wavelength bands can be switched in the light transmission direction by rotating the rotatable holder.

In this embodiment, the holders comprises two rotatable disks 142. The rotatable disks 142 are annular and surrounding the lamp 141 and rotate around the lamp 141, and the optical filters 143 are installed by the rotatable disk 142 along circumferences of the rotatable disks and extends longitudinally. The first optical sheet assembly also includes a rotation adjustment device. The rotation adjustment device includes an adjustment knob 1421, a linkage rod 1422, and a gear 1423. The linkage rod 1422 and the gear 1423 are connected to the adjustment knob 1421 and can rotate together. The gear 1423 and one rotatable disk 142 have external teeth that mesh with each other. The adjusting knob 1421 rotates to take the rotatable disk 142 rotating through the linkage rod 1422 and the gear 1423. The adjustment knob 1421 is generally disposed at a position protruding outside from the main frame 140 to facilitate user operation.

The photon beauty mirror also includes a reflector 144 and a heat dissipation member 145 arranged in the main frame 140. Correspondingly, the reflector 144 is provided with a slot that allows the optical filters 143 to rotate therethrough, and the heat dissipation member 145 is also provided with an opening that allows the optical filters 143 to rotate therethrough. Light from the light source passes through the optical filter 144 in the light transmission direction, and then transmits through the convex lens 146 provided in the main frame 140 and then exits to irradiate skin. The convex lens 146 makes the emitted light more uniform. The opening can be defined by one heat dissipation member 145, or is a space between two heat dissipation members 145; similarly, the slot can be defined by one reflector 144, or is a space between two reflectors 144.

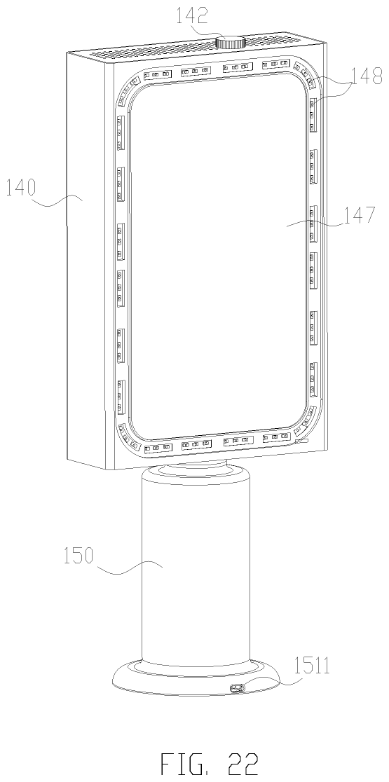

The photon beauty mirror includes a cosmetic lens 147 provided on the main frame 140. The cosmetic lens 147 is arranged on the back of the main frame 140. or the cosmetic lens is detachably arranged in front of the main frame 140. As shown in FIG. 20. the main frame 140 and the cosmetic lens are respectively provided with magnetic components 1022 that cooperate with each other and adsorb each other. The cosmetic lens is attached to the main frame 140 by the magnetic components. A light source 148 is provided around the cosmetic lens 147. As shown in FIG. 22, the light source 148 is provided around the cosmetic lens 147 and connected to the circuit board 155. The light source 148 may use LEDs. A light guide strip 1481 is provided on the outside corresponding to the light source.

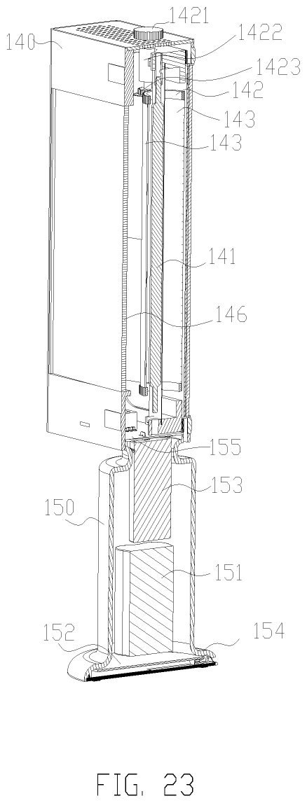

The photon beauty mirror includes a handle 150 connected to the main frame 140, and a rechargeable battery 151 and a charging circuit board 152 provided in the handle 150. The battery 151 is connected to the charging circuit board, as shown in FIG. 23, a capacitor 153 is provided and is electrically connected with the circuit board 155, and the capacitor 153 can trigger the IPL light source to flash under a controlling of the circuit board 155. The battery 151 or an external power supply can supply power to the capacitor 153 which discharges to make IPL light source to flash. The label 154 represents a base.

The photon beauty mirror can be powered by an external power through wires or ports and/or by a battery. A port 1511 can be used for charging the battery or provide power from an external power supply.

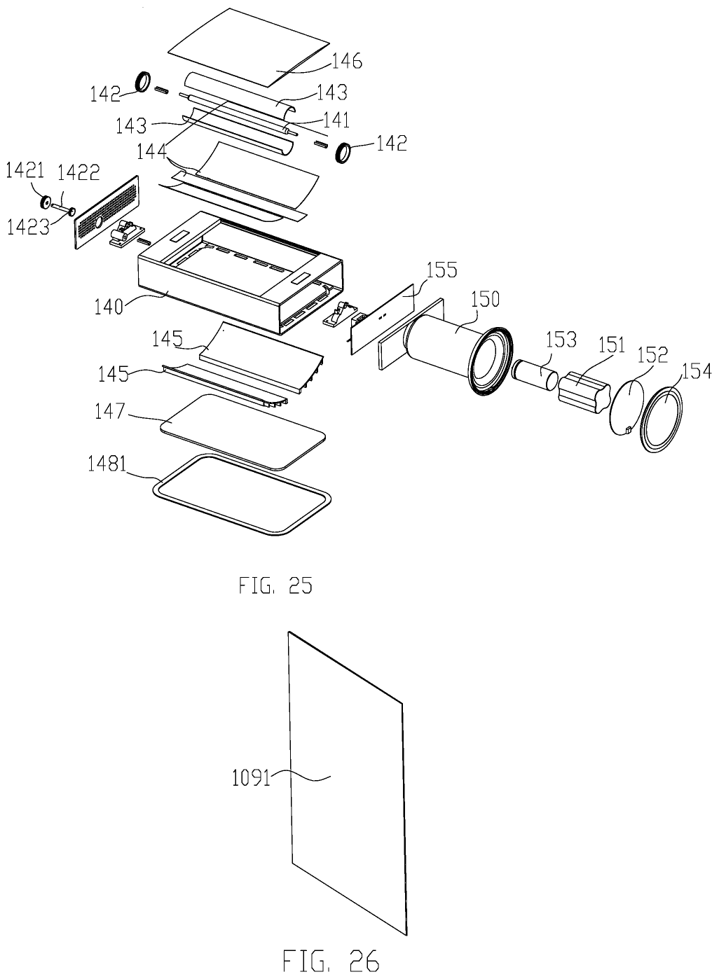

Referring to FIG. 26, which illustrates an optical filter 1091 in accordance with an embodiment of the present invention. The optical filter 1091 comprises an optical substrate coated with a filter film. The filter film can transmit light of some specific wavelength bands, so that the optical filter 1091 can be used as a regular optical filter, and at the same time, the filter film can reflect light of some wavelength bands to obtain a mirror effect, so that the optical filter 1091 can be used as a mirror such as a cosmetic mirror. Light of the specific wavelength bands transmitted through the optical filter 1091 or through the filter film, is generally invisible light, that is, near-infrared light other than sunlight, and such as non-visible light above 700 nm. When visible light below 700 nm irradiates the optical filter 1091, the visible light cannot be transmitted while will be reflected by the optical filter. The filter film of the optical filter 1091 can formed using traditional filter coating process. The optical substrate is made of transparent materials and is a conventional optical substrate.

There are many kinds of the filter film of the optical filter 1091 according to the filter film of different wavelength bands. The optical filters 1091 coated with different color filter films have different light filtering effects. For example, the optical filter 1091 coated with a yellow filter film can pass green light and red light of wavelength bands larger than yellow light, but cannot pass green light and blue light of wavelength bands smaller than yellow light. Another example is that the optical filter 1091 coated with a red-light filter film can pass light of wavelength bands larger than the red light; so green light, yellow light, blue light, and violet light that of wavelength bands smaller than the red light cannot pass through, while red light, near-infrared, mid-infrared and light of wavelength bands equal to or greater than the red light can pass through. In the same way, when the filter film of the optical filter 1091 can only pass light of wavelength bands above the near-infrared of wavelength bands larger than the red light, the optical filter 1091 will appear white or silver, and the visible light in the natural light (sunlight) will not be transmitted through the optical filter 1091. In some specific embodiments, the optical filter 1091 coated with filter film cutting off visible light (with wavelength bands below 700 nm) and prevent the visible light band from being transmitted.

In some embodiment, if the photon beauty mirror comprises the optical filter 1091, when the light source emits flash light with the specific wavelength bands (for example, invisible light that can be used for skin beauty), the optical filter 1091 coated with the filter film of the specific wavelength bands transmits light of the specific wavelength bands. The invisible light of the specific wavelength bands will be transmitted through the optical filter 1091 and irradiate the skin for beauty treatment. When the light source of the photon beauty mirror is turned off, the optical filter 1091 coated with the filter film of the specific wavelength bands will reflect the light of natural light. One side (inside or closer to the photon beauty mirror) of the filter is darker, while the visible light will be reflected on the outer side of the optical filter 1091 and cannot be transmitted so as to increase brightness of the outer side; that is, there is no light from an inter side (inside or closer to the photon beauty mirror) of the filter, while the outer side forms a mirror (i.e., a reflector) because the visible band of natural light is reflected by the optical filter 1091, at this time, the optical filter 1091 can be used as mirrors.

The optical filter 1091 in this embodiment, is not coated with a filter film and a one-way perspective film on either side, but coated with a filter film of specific wavelength bands to obtain both filtering and mirror effects. The structure is simplified, the manufacturing process is simple, and the use effect is good. The optical filter 1091 can be applied in a photon beauty instruments such as photon beauty mirrors, photon skin rejuvenation devices, hair removal devices, import and export beauty devices, or an RF-photon beauty devices.

The optical filter 1091 coated with the filter film of specific wavelength bands is brighter and more delicate than a traditional mirror, because lens of a traditional beauty mirror does not need to transmit light, so the coated film thickness are thicker and particles of the film are larger, which results in a relatively rough surface of the lens, so the refractive index of the reflection is higher, which affects the brightness (such as smooth glass and frosted glass, the brightness of their surface are different); while the optical filter of the photon beauty mirror requires light transmittance, the filter film of the optical filter 1091 has a more delicate molecular structure, lower refractive index and higher reflectivity, which results in higher brightness.

In some embodiments, the photon beauty mirror using the optical filter 1091, includes a main frame, and the filter 1091 is disposed in or on the main frame.

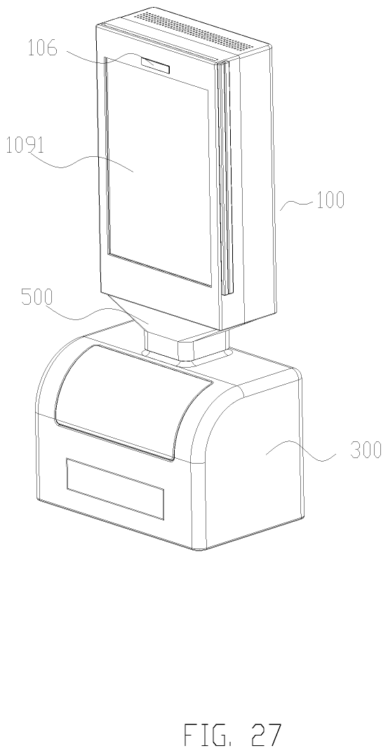

Referring to FIG. 27. the optical filter 1091 coated with a filter film of specific wavelength bands is applied to a photon beauty mirror, which comprises a base 300 and a main frame 100. The optical filter 1091 can be installed to the main frame 100 in a plug-in manner.

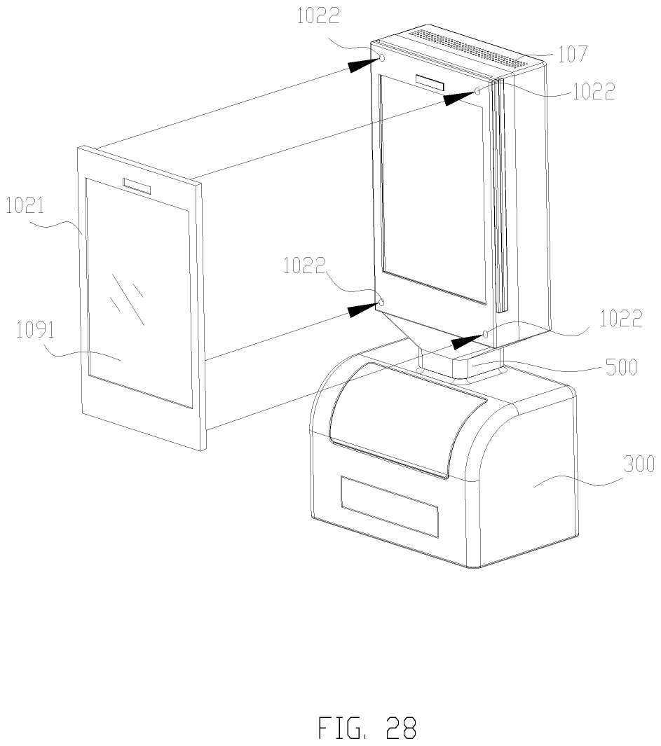

Referring to FIG. 28, the optical filter 1091 coated with a filter film of specific wavelength bands is applied to a photon beauty mirror, where the optical filter 1091 is attached to the main frame 100 by magnetic components 1022.

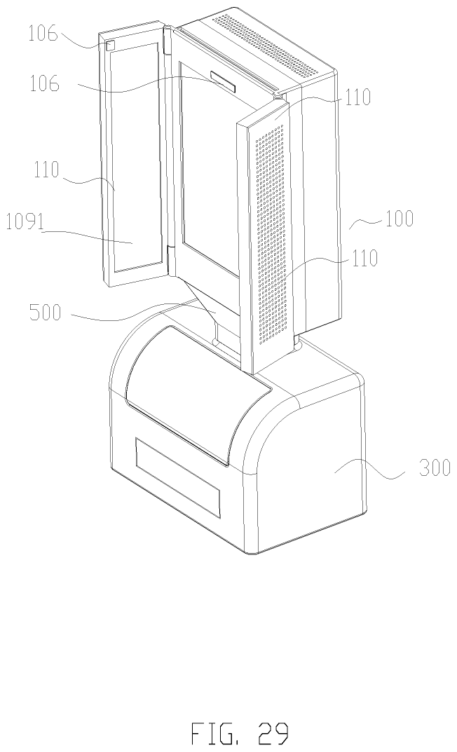

The photon beauty mirror of FIG. 29 includes a base 300, a main frame 100 and auxiliary frames 110. The filter 1091 coated with the filter film of specific wavelength bands can be installed to the main frame 100 and/or the auxiliary frames 110.

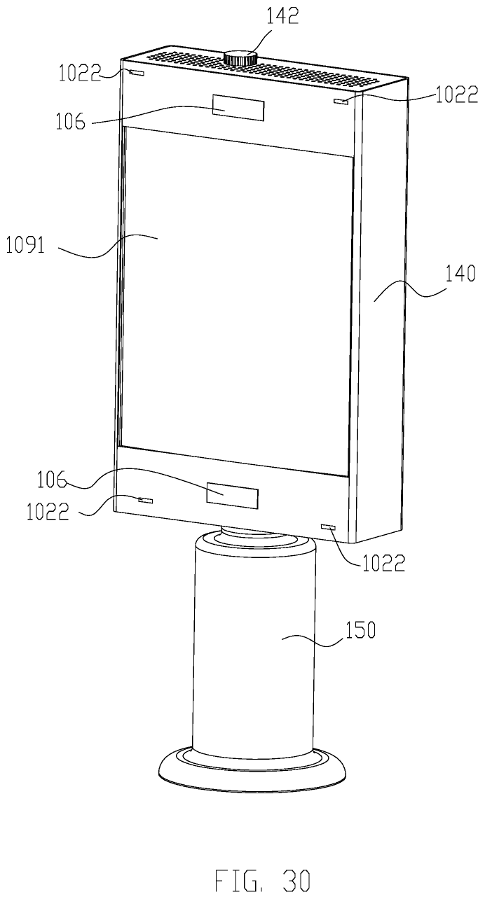

Referring to FIG. 30, the optical filter 1091 coated with a filter film of specific wavelength bands is applied to a photon beauty mirror, which comprises a handle 150 and a main frame 140. The optical filter 1091 can be installed to the main frame 100 in a plug-in manner or by magnetic components 1022.

The technical features of the above embodiments can be combined arbitrarily. In order to make the description simple, all possible combinations of the technical features in the above embodiments are not described. However, as long as there is no contradiction in the combination of these technical features It is considered to be the range described in this specification.

The above examples only represent several embodiments of the present invention, and the descriptions thereof are specific and detailed, but should not be construed as a limitation on the scope of the invention patent. It should be pointed out that for those of ordinary skill in the art, without departing from the concept of the present invention, several modifications and improvements can also be made, which all belong to the protection scope of the present invention. Therefore, the protection scope of the patent of the present invention should be subject to the appended claims.

What is claimed is:

1. A photon beauty mirror, comprising:
a main frame;
a first light source assembly disposed in the main frame, and comprising a first light source for emitting light;
a circuit board disposed in the main frame and electrically connected with the first light source assembly; and
a first optical sheet assembly installed to the main frame comprising one or more optical filters for filtering light from the light source to obtain a preset wavelength bands;
wherein at least one optical filter is a special optical filter; the special optical filter comprising:
an optical substrate coated with a filter film of a preset wavelength bands on one side and coated with a one-way perspective film on an opposite side of the optical sheet, light from the light source penetrate the one-way perspective film and the optical substrate, and light of the preset wavelength bands passes through the filter film for beauty treatment; the one-way perspective film transmits incident light on one side and reflects visible light on the other side, and the side reflecting incident visible light gives a mirror effect; or
an optical substrate coated with a filter film, where near infrared light or non-visible light above 700 nm transmit through the filter film for beauty treat, and the filter film reflects visible to obtain a mirror effect.

2. The photon beauty mirror as claimed in claim 1, wherein more first optical sheet assemblies are provided; the first optical sheet assemblies or optical filters are detachably and replaceably installed to the main frame each for filtering light from the first light source; and the optical sheet assemblies each comprise a regular optical filter, or the special filter film of different wavelength bands, or a lens of a reflecting mirror.

3. The photon beauty mirror as claimed in claim 2, wherein the regular optical filter or the special optical filter or the lens is equipped in a holder.

4. The photon beauty mirror as claimed in claim 3, wherein the main frame defines one or more slots for receiving the first optical sheet assembly, and the optical sheet assembly or the optical filter is replaceably installed to the main frame by inserting into the slot.

5. The photon beauty mirror as claimed in claim 3, wherein the optical sheet assembly is replaceably attached to the main frame by magnetic components installed in the main frame and the holder.

6. The photon beauty mirror as claimed in claim 1, further comprising one or two auxiliary frames each connected to either side of the main frame; wherein each auxiliary frame is equipped with a second light source assembly and a second optical sheet assembly; the second light source assembly comprises a second light source, and the second optical sheet assembly comprises the special optical filter or a regular optical filter; light from the second light source irradiates through the special optical filter or the regular optical filter for beauty treatment.

7. The photon beauty mirror as claimed in claim 6, wherein the second optical sheet assembly is installed to the auxiliary frame by inserting into a slot defined in the auxiliary frame, or by magnetic components disposed in the auxiliary frame and the second optical sheet assembly, or fixedly installed to the auxiliary frame; the auxiliary frame defines heat dissipation holes; and the main frame defines heat dissipation holes.

8. The photon beauty mirror as claimed in claim 6, wherein the auxiliary frame is rotatably connected to either side of the main frame through a rotating shaft; the auxiliary frame rotates around either side of the main frame to open or close a front side of the main frame; and a rotatable angle of the auxiliary frame is adapted for different widths of user's face to be treated.

9. The photon beauty mirror as claimed in claim 1, wherein the first light source assembly comprises a rotatable holder fixing an end of the one or more optical filters; the optical filter is in a shape of a cylinder or are fixed by the rotatable holder to form a cylinder; the light source is longitudinally arranged in the cylinder; the rotatable holder takes the cylinder to rotate around the light source so as to change a position of the one or more optical filters.

10. The photon beauty mirror as claimed in claim 9, wherein the first light source assembly comprises at least one reflector and at least one heat dissipation member; the at least one reflector is disposed behind the light source; the at least one heat dissipation member is installed behind the reflector; the at least one reflector forms a slot that allows the optical filter to rotate therethrough, the at least one heat dissipation member forms an opening that allows the optical filter to rotate therethrough.

11. The photon beauty mirror as claimed in claim 10, wherein the optical filter is formed by coating filter films on a cylindrical optical substrate, the filter films have different wavelength bands in zones, each zone is formed on the cylinder peripheral wall along a longitudinal direction, different zones of different wavelength bands rotate by rotating the rotatable holder, the light source is longitudinally arranged within the optical filter; the rotatable holder takes the optical filter to rotate around the light source so as to switch different zone of the optical filter in a light transmission direct of the light source; the optical filter rotates around the light source to change positions of the zones; and at least one zone forms the special optical filter.

12. The photon beauty mirror as claimed in claim 9, wherein the one or more optical filters rotate around the light source to switch from a first position to a second position; and the first position is in a light transmission direct; light from the light source passes through the optical filter switched in the first position to obtain a preset wavelength bands for beauty treatment.

13. The photon beauty mirror as claimed in claim 12, wherein the rotatable holder is an annular disk, the one or more optical filters are installed by the annular disk along circumferences of the annular disks and extends longitudinally.

14. The photon beauty mirror as claimed in claim 13, comprising a rotation adjustment device, wherein the rotation adjustment device comprises an adjustment knob, a linkage rod, and a gear; the linkage rod and the gear are connected to the adjustment knob and can rotate together; the gear and one annular disk have external teeth that mesh with each other; the adjusting knob rotates to take the rotatable disk rotating through the linkage rod and the gear.

15. The photon beauty mirror as claimed in claim 14, wherein the adjustment knob is disposed at a position protruding outside from the main frame to facilitate user operation.

16. The photon beauty mirror as claimed in claim 1, comprising a handle connected to the main frame, wherein a battery and/or a capacitor electrical connected to the circuit board are installed in the handle.

17. The photon beauty mirror as claimed in claim 1, wherein the first light source assembly comprises a reflector and a convex lens; the reflector is disposed behind the light source, and the convex lens is disposed between the light source and the optical filter, whereby light from the light source is transmitted through the convex lens to irradiate skin evenly.

18. The photon beauty mirror as claimed in claim 6, wherein the second light source assembly comprises a reflector and a convex lens; the reflector is disposed behind the second light source, and the convex lens is disposed between the second light source and the optical filter, whereby light from the second light source is transmitted through the convex lens to irradiate skin evenly.

19. The photon beauty mirror as claimed in claim 1, further comprising goggles to protect the user's eyes; wherein the goggles and the main frame are respectively provided with sensors for detecting a distance between the goggles and the light source so that the circuit board controls turning on/off the light source; the goggles has a positioning block for limiting a distance between the goggles and the main frame; the photon beauty mirror comprises a base and a head support frame; the base is provided with an accommodation cavity for storing the goggles and an accommodation cavity for storing the head support frame; the head support frame is used to support user's head to facilitate positioning the face; a connecting frame is provided between the base and the main frame; the connecting frame defines a ventilation channel in air communication with the main frame; and a fan is provided in the connecting frame and in air communication with the ventilation channel.

20. The photon beauty mirror as claimed in claim 1, wherein the main frame defines heat dissipation holes and is provided with heat dissipation member therein; the main frame defines air channels in air communication with the dissipation holes, where one air channel passes a surface of the optical filter, one air channel passes through the light source; and one air channel passes through a reflector and/or a heat dissipation member.
